(19) United States
(12) Patent Application Publication
(10) Pub. No.: US 2016/0309916 A1
(43) Pub. Date: Oct. 27, 2016

(54) INFANT BED ATTACHMENT SYSTEM

(71) Applicant: KONINKLIJKE PHILIPS N.V., Eindhoven (NL)

(72) Inventors: Manju Catherine POTHEN, BANGALORE (IN); Shivi KAPIL, BANGALORE (IN)

(21) Appl. No.: 15/105,060

(22) PCT Filed: Jan. 8, 2015
(86) PCT No.: PCT/IB2015/050141

§ 371 Date: Jun. 16, 2016

Related U.S. Application Data
(60) Provisional application No. 61/928,131, filed on Jan. 16, 2014.

(51) Int. Cl. A47D 15/00; A61G 13/10; A61G 10/04; A61B 5/11; A61B 5/113; A61G 11/00; A61B 5/00

(52) U.S. Cl. CPC A47D 15/00; A61G 11/006; A61G 13/101; A61B 5/0077; A61B 5/1128; A61B 5/1135; A61G 10/04

(57) ABSTRACT

The present disclosure pertains to an infant sleeping surface attachment system configured to facilitate vital signs monitoring of, and/or delivering oxygen to, an infant laying on an infant sleeping surface. The system is configured to engage a vital signs monitoring device so the vital signs monitoring device has a view of the infant sleeping surface. The system has a retracted position and a closed position. The closable cavity has a mounting apparatus for engaging a vital signs monitoring device inside the cavity. The mounting apparatus is configured such that the vital signs monitoring device maintains a field of view on a region of interest and minimizes the possibility of occluding the view of the vital signs monitoring device. Thus, it is possible to continuously monitor of the infant from inside the cavity without having any glass/plastic material (e.g., the wall of a typical oxyhood) obstructing the field of view.

### BACKGROUND

1. Field

The present disclosure pertains to an infant sleeping surface attachment system configured to engage a vital signs monitoring device so the vital signs monitoring device has a view of the infant sleeping surface. When necessary, a flexible and moveable portion of the infant sleeping surface attachment system is configured to close to deliver oxygen to an infant laying on the infant sleeping surface without obstructing the field of view of the vital signs monitoring device.

2. Description of the Related Art

Typically, neonatal intensive care units (NICU) are fragile environments consisting of sick preterm and term neonatal infants. Vital signs monitoring of these neonates is usually done using invasive methods with attached probes, sensors, electrodes, and/or other devices. Non-invasive monitoring methods minimize risks associated with invasive monitoring but their use is limited. Camera based monitoring is one such method. A camera may be placed on and/or near the bed of a neonate. Physiological parameters are determined from processed camera images. The placement of such a camera is very important in order to accurately measure vital signs and to replace invasive monitoring methods. There are several constraints on this placement due to NICU equipment, activities, and procedures. The NICU equipment, activities, and procedures (e.g., caregivers conducting procedures on the baby, treatment equipment, tubes, blankets) may occlude the view of the camera. Fore example, during the use of an oxyhood for neonates who need supplemental oxygen, an oxyhood is placed over the face and neck of an infant and oxygen is pumped into the oxyhood. After multiple uses and subsequent cleanings, the surface of the oxyhood is typically scratched and damaged, which makes visibility through the hood very difficult and makes camera based monitoring impossible.

### SUMMARY

Accordingly, one or more aspects of the present disclosure relate to an infant sleeping surface attachment system configured to facilitate vital signs monitoring of, and/or delivering oxygen to, an infant laying on an infant sleeping surface. The system comprises a hood and a cover portion. The hood comprises a fixed portion. The fixed portion includes a mounting apparatus. The fixed portion is configured to be held engaged in a fixed position relative to the infant sleeping surface. The fixed portion forms a cavity having an open mouth formed in part by the infant sleeping surface. The mounting apparatus is configured to engage a vital signs monitoring device. The mounting apparatus is coupled to the fixed portion inside the cavity. The mounting apparatus is configured to carry the vital signs monitoring device so the vital signs monitoring device has a view of the infant sleeping surface. The cover portion is movably coupled with the fixed portion. The cover portion is movably coupled with the fixed portion such that the cover portion moves with respect to the fixed portion between a retracted position and a closed position. In the closed position, the cover portion covers the open mouth of the fixed portion. In the retracted position, the cover portion permits physical access to the cavity.

Another aspect of the present disclosure relates to a method for facilitating vital signs monitoring of, and/or delivering oxygen to, an infant laying on an infant sleeping surface with an infant sleeping surface attachment system. The system comprises a hood and a cover portion. The hood comprises a fixed portion and a mounting apparatus. The method comprises forming a cavity with the fixed portion that is held engaged in a fixed position relative to the infant sleeping surface. The cavity has an open mouth formed in part by the infant sleeping surface. The method comprises engaging a vital signs monitoring device with the mounting apparatus. The mounting apparatus is coupled to the fixed portion inside the cavity. The mounting apparatus is configured to carry the vital signs monitoring device so the vital signs monitoring device has a view of the infant sleeping surface. The method comprises movably coupling the cover portion with the fixed portion such that the cover portion moves with respect to the fixed portion between a retracted position and a closed position. In the closed position, the cover portion covers the open mouth of the cavity. In the retracted position, the cover portion permits physical access to the cavity.

Still another aspect of present disclosure relates to an infant sleeping surface attachment system configured to facilitate vital signs monitoring of, and/or delivering oxygen to, an infant laying on an infant sleeping surface. The system comprises means for forming a cavity that is held engaged in a fixed position relative to the infant sleeping surface. The cavity has an open mouth formed in part by the infant sleeping surface. The system comprises means for engaging a vital signs monitoring device. The means for engaging is coupled to the means for forming a cavity inside the cavity. The means for engaging is configured to carry the vital signs monitoring device so the vital signs monitoring device has a view of the infant sleeping surface. The system comprises means for covering the open mouth of the cavity and permitting physical access to the cavity. The means for covering and permitting is configured to move with respect to the means for forming a cavity between a retracted position and a closed position. In the retracted position, the means for covering and permitting permits physical access to the cavity. In the closed position, the means for covering and permitting covers the open mouth of the cavity.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is an illustration of an infant sleeping surface attachment system.

FIG. 2 illustrates a side view of the infant sleeping surface attachment system.

FIG. 3 illustrates a collapsible embodiment of the infant sleeping surface attachment system.

FIG. 4 illustrates the system on a typical warmer in a NICU.

FIG. 5 illustrates the system in use at home and/or in a ward at a hospital.

FIG. 6 illustrates a method for facilitating vital signs monitoring of an infant laying on an infant sleeping surface with an infant sleeping surface attachment system.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

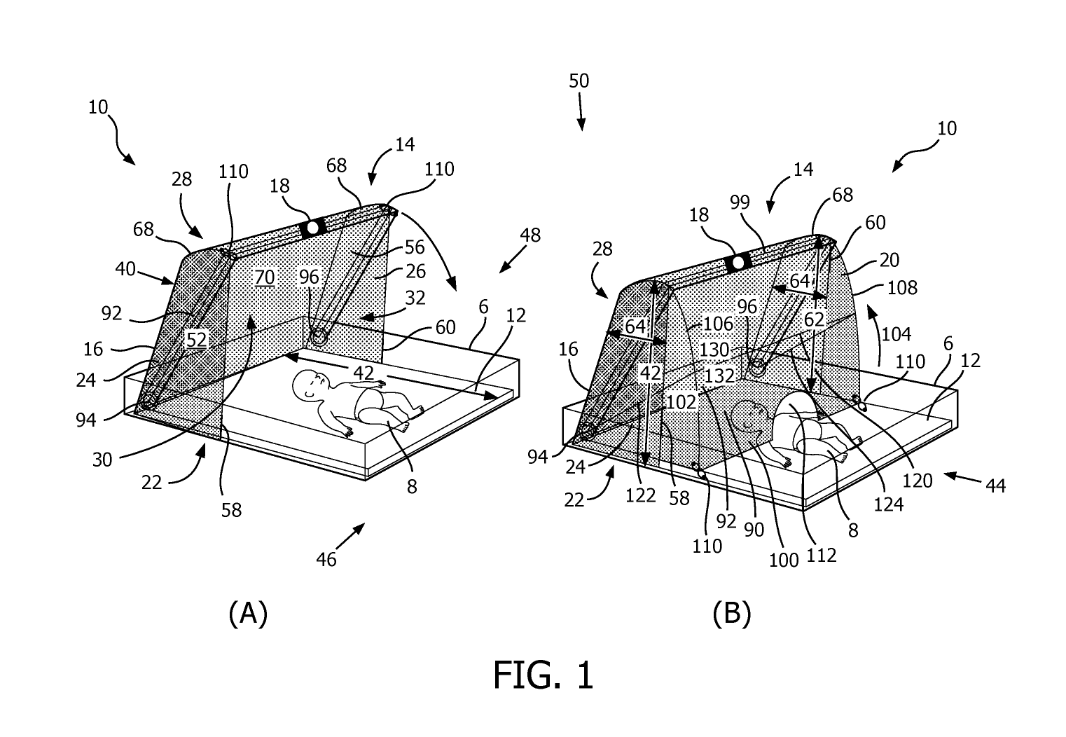

FIG. 1 is an illustration of an infant sleeping surface attachment system 10 configured to deliver oxygen to an infant 8 laying on an infant sleeping surface 12. System 10 is configured to engage a vital signs monitoring device so the vital signs monitoring device has a view of infant sleeping surface 12 before, during, and/or after oxygen delivery to infant 8. FIG. 1A illustrates system 10 in a retracted position. FIG. 1B illustrates system 10 in a closed position. FIG. 1A and FIG. 1B both illustrate system 10 within a NICU bed 6, but this is not intended to be limiting.

System 10 overcomes constraints of typical oxyhoods by forming a closable cavity 30 over infant sleeping surface 12. The closable cavity has a mounting apparatus 18 for engaging a vital signs monitoring device inside cavity 30. Mounting apparatus 18 is configured such that the vital signs monitoring device maintains a field of view on a region of interest (e.g., the head and chest of infant 8, sleeping surface 12) and minimizes the possibility of occluding the view of the vital signs monitoring device. With system 10 in the retracted position (FIG. 1A), system 10 acts as a camera stand in close proximity to infant 8. This may provide a closer view of body parts of infant 8 for heart rate monitoring, an angled view of infant 8 for monitoring chest movements required for respiratory rate monitoring, and/or other advantages, while still allowing physical access to infant 8 by caregivers for normal NICU activities. When system 10 is in the closed position (FIG. 1B), there is no presence of any other glass/plastic (e.g., an incubator box, typical oxyhood) between the vital signs monitoring device and infant 8.

In some embodiments, system 10 includes a hood 14, a cover portion 20, and/or other components. Hood 14 includes a fixed portion 16 and/or other components. Fixed portion 16 includes a mounting apparatus 18 and/or other components. In some embodiments, hood 14 and cover portion 20 are at least partially formed from substantially transparent materials so infant 8 may be viewed through hood 14 and/or cover portion 20 when system 10 is in the closed position.

Fixed portion 16 is configured to be held engaged in a fixed position relative to infant sleeping surface 12. Fixed portion 16 forms cavity 30. Cavity 30 has an open mouth 32 formed in part by infant sleeping surface 12. In some embodiments, open mouth 32 may have a substantially rectangular shape. In some embodiments, open mouth 32 may have a non-rectangular shape. Fixed portion 16 is configured to at least partially enclose a first end 40 of infant sleeping surface 12 such that open mouth 32 of cavity 30 faces an opposite second end 44 of infant sleeping surface 12. Fixed portion 16 is configured to at least partially enclose first end 40 of infant sleeping surface 12 such that open mouth 32 is substantially perpendicular to a length 42 of infant sleeping surface 12. In some embodiments, fixed portion 16 may partially enclose first end 40 without substantially deforming infant sleeping surface 12. Substantially deforming infant sleeping surface 12 may include may include deforming infant sleeping surface 12 to a point where it is no longer safe for infant 8 to lay and/or sleep on infant sleeping surface 12.

Fixed portion 16 includes a base panel 22, a first side panel 24, a second side panel 26, an end panel 28, and/or other components. Base panel 22 is configured to be positioned under first end 40 of infant sleeping surface 12. In some embodiments, base panel 22 is a substantially planar surface that slides under infant sleeping surface 12. In some embodiments, base panel 22 may have a rectangular form factor and/or other form factors. Base panel 22 extends from a first side 46 of infant sleeping surface 12 to a second side 48 of infant sleeping surface 12. Base panel 22 extends from first end 40 of infant sleeping surface 12 toward second end 44. Base panel 22 extends far enough from first end 40 toward second end 44 so that fixed portion 16 removably engages infant sleeping surface 12 and infant sleeping surface 12 holds fixed portion 16 and/or cavity 30 engaged in a fixed position at first end 40.

In some embodiments, the weight, form factor, and/or other properties of infant sleeping surface 12 hold fixed portion 16 and/or cavity 30 engaged in the fixed position. In some embodiments, fixed portion 16 may be held engaged in the fixed position by something other than sleeping surface 12. For example, fixed portion 16 may be held engaged in a fixed position by a bedframe, at least a portion of a NICU infant enclosure, the weight and/or structure of fixed portion 16, clamps, hooks, straps, clips, adjustable attachment fixtures and/or locking mechanisms, and/or other devices.

First side panel 24 is coupled with base panel 22 at first side 46. First side panel 24 extends from base panel 22 to a third side 50 of fixed portion 16. First side panel 24 forms a substantially planar first side surface 52 of cavity 30 on first side 46 of infant sleeping surface 12. Side surface 52 may be substantially parallel to length 42 of infant sleeping surface 12. An edge 58 of first side surface 52 toward second end 44 may define a portion of open mouth 32 on first side 46.

Second side panel 26 opposes first side panel 24. Second side panel 24 is coupled with base panel 22 at second side 48. Second side panel 26 extends from base panel 22 to third side 50 of fixed portion 16. Second side panel 26 forms a substantially planar second side surface 56 of cavity 30 on second side 48 of infant sleeping surface 12. Side surface 56 may be substantially parallel to length 42 of infant sleeping surface 12. Second side surface 56 faces first side surface 52. An edge 60 of second side surface 56 toward second end 44 may define a portion of open mouth 32 on second side 48.

First side panel 24 and second side panel 26 have a length 62 extending from base panel 22 to third side 50. First side panel 24 has a width 64 extending from edge 58 to first end 40. Second side panel 26 has width 64 extending from edge 60 to first end 40. Width 64 decreases as first side panel 24 and/or second side panel 26 extend away from base panel 22. In some embodiments, at third side 50, first side panel 24 and second side panel 26 have corresponding arcuately shaped edges 68 (as shown in FIGS. 1A and 1B).

End panel 28 is coupled with base panel 22, first side panel 24 and second side panel 26 at first end 40 of infant sleeping surface 12. End panel 28 is substantially perpendicular to length 42 and forms an end surface 70 of cavity 30 at first end 40. End panel 28 extends from base panel 22 at end 40 toward second end 44 and third side 50, forming an acute angle with infant sleeping surface 12. End panel 28 is coupled to first side panel 24 and second side panel 26 such that the acute angle formed with infant sleeping surface 12 corresponds to decreasing width 64. In some embodiments, at least a portion of end panel 28 has an arcuate shape that corresponds to arcuately shaped edges 68 of first side panel 24 and second side panel 26.

Fixed portion 16 includes mounting apparatus 18. Mounting apparatus 18 is configured to engage a vital signs monitoring device. In some embodiments, the vital signs monitoring device is and/or includes a vital sign monitoring camera, and/or other devices. In some embodiments, the vital signs monitoring device and/or the vital sign monitoring camera includes and/or is associated with audio data recording devices, a lighting arrangement (e.g., as per NICU standard guidelines), other visual data recording devices, and/or other equipment. Mounting apparatus 18 is coupled to fixed portion 16 inside cavity 30. Mounting apparatus 18 is configured to carry the vital signs monitoring device so the vital signs monitoring device has a view of infant sleeping surface 12. In some embodiments, mounting apparatus 18 is configured such that the vital signs camera has a view of the face of infant 8 laying on infant sleeping surface 12. In some embodiments, mounting apparatus 18 is configured such that the vital signs monitoring camera has an angled view of the chest of infant 8 while infant 8 lays on sleeping surface 12.

Mounting apparatus 18 may include one or more of an adhesive, a buckle (e.g., a quick release buckle), a screw and/or a threaded hole (e.g., similar to and/or the same as a standard camera tripod mount), a pivot arm, a fastener (e.g., hook and eye), a tether, a cam, a clamp, a clip, a tightening device, a suction cup, a vibration dampening device, molded with fixed portion 16 as a continuous piece, and or other devices for engaging various vital signs monitoring devices. Mounting apparatus 18 may include mounting features that correspond to mounting features on vital signs monitoring devices. Mounting apparatus 18 may be configured to removably engage a vital signs monitoring device. Mounting apparatus 18 may be configured to removably engage one or more vital signs monitoring devices at the same time. Mounting apparatus 18 may be configured to slidably engage vital signs monitoring devices. In some embodiments, mounting device 18 may be coupled with fixed portion 16 via adhesive, screws, clips, clamps, buckles, sewing, bonding, and/or other coupling devices, techniques, and/or methods. Mounting apparatus 18 is coupled with fixed portion 16 such that mounting apparatus 18 does not interfere with folding and/or collapsing of system 10.

FIG. 1A and FIG. 1B illustrate mounting apparatus 18 coupled with fixed portion 16 on end surface 70 inside cavity 30. Mounting apparatus 18 is coupled with fixed portion 16 on end surface 70 at or near a centerline of end surface 70, at or near third side 50 (e.g., on the portion of end panel 28 that has an arcuate shape). This is not intended to be limiting. In some embodiments, mounting apparatus 18 may be coupled with surface 52, surface 56, and/or any other part of fixed portion 16 inside cavity 30 provided the vital signs monitoring device has a view of infant sleeping surface 12 that facilitates monitoring of infant 8.

The location where mounting apparatus 18 is coupled with fixed portion 16 may be determined based on the structure of a NICU bed, procedures performed by caregivers that may occlude a camera's (for example) field of view, accessories contained within the bed that may occlude a camera's field of view, possible infant movement, and/or other factors. For example, mounting apparatus 18 may be positioned so a vital signs camera has a field of view that includes the entirety of sleeping surface 12 (e.g., so that an infant may move anywhere on the sleeping surface and still be in view of the camera). Mounting apparatus 18 may be positioned so the vital signs monitoring device does not hinder any of the regular activities of NICU caregivers while at the same providing accurate vital signs monitoring. The region of interest for heart rate monitoring is typically the face of infant 8 and for respiratory rate monitoring, the chest movements of infant 8. Mounting apparatus 18 may be coupled with fixed portion 16 such that when the vital signs monitoring device is engaged, the camera, for example, is in close proximity to the region of interest (e.g., the chest and body of infant 8).

Cover portion 20 is movably coupled with fixed portion 16. In some embodiments, cover portion 20 is slidably coupled with fixed portion 16. Cover portion 20 moves and/or slides with respect to fixed portion 16 between a retracted position (shown in FIG. 1A) and a closed position (shown in FIG. 1B). In the closed position, cover portion 20 covers open mouth 32 of fixed portion 16. In the retracted position, cover portion 20 permits physical access to cavity 30. In some embodiments, cover portion 20 comprises a flap 90, a support frame 92, bearings 94 and 96, and/or other components.

Flap 90 is configured to deploy to cover open mouth 32 of fixed portion 16. Flap 90 may be formed from a substantially transparent, flexible material. The transparency of flap 90 may allow caregivers to maintain visual contact with the face of infant 8 when flap 90 is closed. The flexibility of flap 90 may make it less bulky and easy to extend into the closed position by caregivers. In some embodiments, flexible flap 90 includes an end flap 100, a first side flap 102, a second side flap 104, and/or other portions. As shown in FIG. 1A, in the retracted position, flap 90 is configured to be withdrawn into cavity 30. Flap 90 is configured to be withdrawn into cavity 30 without interrupting the vital signs monitoring device held by mounting apparatus 18 (e.g., without blocking the view of infant sleeping surface 12). With flap 90 withdrawn into cavity 30, no separate space is required to store flap 90. This is not the case with typical oxyhoods. Typical oxyhoods are bulky and require extra storage space.

As shown in FIG. 1B, in the closed position, first side flap 102 and second side flap 104 are generally wedge shaped having triangular corners located near base panel 22 and end panel 28. First side flap 102 is substantially parallel to first side panel 24 and positioned at least partially inside cavity 30 along surface 52 and partially outside cavity 30. First side flap 102 has an arcuate edge 106 toward second end 44. Second side flap 104 is substantially parallel to second side panel 26 and positioned at least partially inside cavity 30 along surface 56 and partially outside cavity 30. Second side flap 104 has an arcuate edge 108 toward second end 44 that corresponds to arcuate edge 106. Arcuate edges 106 and 108 extend from fixed portion 16 at third side 50 toward infant sleeping surface 12 and/or base panel 22.

End flap 100 has an arcuate shape. End flap 100 is fixed to arcuate edges 106 and 108 and encloses cavity 30. In some embodiments, an edge 99 of end flap 100 is removably coupled with fixed portion 16 at or near third side 50. In the closed position (e.g., FIG. 1B), end flap 100 is removably coupled with infant sleeping surface 12 and/or some other portion of a bed and/or other structure holding infant 8. End flap 100 is removably coupled with fixed portion 16 and/or infant sleeping surface 12 via attachment mechanisms 110. Attachment mechanisms 110 include clamps, straps, fabric loops, hook and eye (e.g., Velcro) fasteners, clips, and/or other removable fixation devices.

In some embodiments, fixed portion 16 may include corresponding attachment features configured to be engaged by attachment mechanisms 110. In some embodiments, the attachment features may be located on third side 50 of fixed portion 16 near first side 46 and/or second side 48, and/or in other locations. In some embodiments, infant sleeping surface 12 and/or another portion of a bed and/or other structure holding infant 8 may include corresponding attachment features configured to be engaged by attachment mechanisms 110 (e.g., on first side 46 and/or second side 48 of infant sleeping surface 12). In the retracted position (e.g., FIG. 1A), attachment mechanisms 110 are removably coupled with fixed portion 16. In the closed position, attachment mechanisms are removably coupled with infant sleeping surface 12. For example, when an oxyhood is required, a caregiver may pull attachment mechanisms 110 from attachment points on fixed portion 16 and then attach attachment mechanisms 110 to infant sleeping surface 12 to secure cover portion 20 in place during oxygen therapy. As shown in FIG. 1B, flap 90 includes an opening 112 that fits around the neck and/or body of infant 8 when cover portion 20 is in the closed position.

Support frame 92 is configured to support flap 90. Support frame 92 includes a cross-member 120, a first side member 122, a second side member 124, and/or other components. Members 120, 122, and 124 are substantially rigid members configured to support flap 90. Members 120, 122, and 124 may be formed from one or more of a metal, a polymer, a ceramic, a composite, and/or any other light-weight, rigid materials. Members 120, 122, and 124 may be removably and/or fixedly coupled together to form a single generally U-shaped member. Cross-member 120 spans a width of end flap 100. The width of end flap 100 is substantially perpendicular to length 42 of infant sleeping surface 12. First side member 122 extends from first side 46 of cross-member 120 to bearing 94 along first side flap 102. Second side member extends from second side 48 of cross-member 120 to bearing 96 along second side flap 104. Support frame 92 is configured to support flap 90 such that end flap 100 has a generally arcuate shape in the closed position. Support frame 92 gives end flap 100 an arcuate shape because first side member 122 and second side member 124 are longer than the widest portions of first side panel 24 and second side panel 26 (e.g., the portions nearest base panel 22) and because cross-member 120 is coupled to end panel 28 in a position that is approximately half way between base panel 22 and third end 50 when cover portion 20 is in the closed position. Support frame 92 may rotate because an end of first side member 122 is coupled with bearing 94 and an end of second side member 124 is coupled with bearing 96. Support frame 92 may rotate into cavity 30 (e.g., toward third end 50) when cover portion 20 is in the retracted position.

Support frame 92 may divide flap 90 into sectors. FIG. 1B illustrates two sectors 130 and 132. Dividing flap 90 into sectors may make it easier for caregivers and/or other users to deploy cover portion 20. For example, during retraction into cavity 30, sector 132 may fold onto and/or over sector 130. During closing just prior to oxygen therapy, sector 132 may unfold from and/or off of sector 130 as flap 90 is extended toward infant sleeping surface 12. Dividing flap 90 into sectors may make it easier to retract cover portion into cavity 30 without blocking the view of a vital signs monitoring device monitoring infant 8. Dividing flap 90 into sectors may give flap 90 a generally arcuate shape in the closed position. Dividing flap 90 into sectors may make flap 90 easier to clean, may create a desired volume of enclosed cavity 30 for oxygen therapy, may better accommodate infant movement when cover portion 20 is in the closed position, and/or have other advantages. In some embodiments, cross-member 120, first side member 122, and second side member 124 are removably coupled with, and/or fixedly coupled with flap 90. For example, members 120, 122, and/or 124 may be sewn to flap 90, clipped to flap 90, coupled with flap 90 using an adhesive, and/or coupled via other methods.

Support frame 92 is the only support fame illustrated in FIG. 1B. This is not intended to be limiting. System 10 may include two or more support frames that divide flap 90 into any number of sectors. The two or more support frames may be coupled with bearings 94 and 96 and be deployed in any way that allows system 10 to function as described herein.

Bearings 94 and 96 are configured to facilitate rotation and/or other movement of cover portion 20 between the retracted position and the closed position. In some embodiments, bearings 94 and 96 include ball bearings, hinges, locking/unlocking mechanisms (e.g., similar to door locking mechanisms), and/or other devices that facilitate movement of cover portion 20 between the retracted position and the closed position. Bearing 94 is coupled with first side panel 24 and first side member 122 near base panel 22 and end panel 28. Bearing 96 is coupled with second side panel 26 and second side member 124 near base panel 22 and end panel 28. In some embodiments, bearing 94 is coupled with first side panel 24 on surface 52. In some embodiments, bearing 96 is coupled with second side panel 26 on surface 56. In some embodiments, bearing 94 is removably coupled with first side member 122. In some embodiments, bearing 96 is removably coupled with second side member 124. Bearings 94 and 96 are removably coupled with side members 122 and 124 so that cover portion 20 may be decoupled from fixed portion 16 for cleaning and/or other activities. In some embodiments, cover portion 20 may be decoupled from fixed portion 16 when system 10 is only needed as a camera stand, for example, during vital signs monitoring of infant 8.

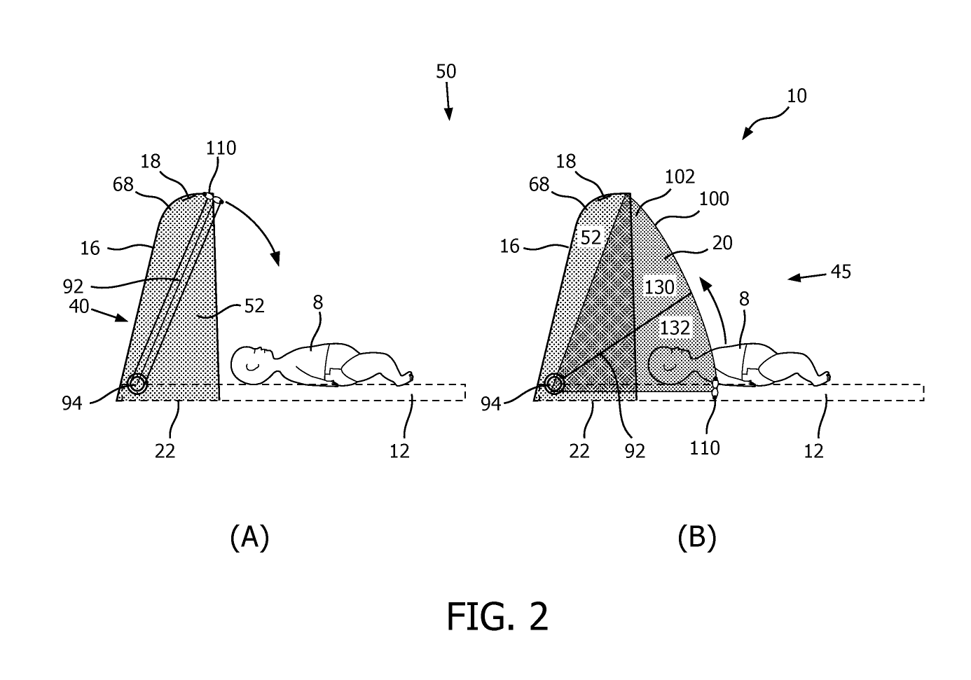

FIG. 2 illustrates a side view of infant sleeping surface attachment system 10. FIG. 2A illustrates a side view of system 10 in a retracted position and FIG. 2B illustrates a side view of system 10 in the closed position. In the retracted position (FIG. 2A), cover portion 20 (shown folded up) is coupled with fixed portion 16 at or near third end 50. Cover portion 20 may be rotated to the closed position (FIG. 2B) by a caregiver and/or other users via bearing 94 (and bearing 96 which is not shown). In the closed position, support frame 92 maintains the arcuate shape of end flap 100. Flap 90 may be removably coupled to infant sleeping surface 12 via attachment mechanisms 110. Mounting apparatus 18 is positioned (whether system 10 is in the retracted position or the closed position) so a vital signs camera has a field of view that includes a region of interest of on infant 8 and so the vital signs monitoring device does not hinder any of the regular activities of NICU caregivers (e.g., while at the same providing accurate vital signs monitoring).

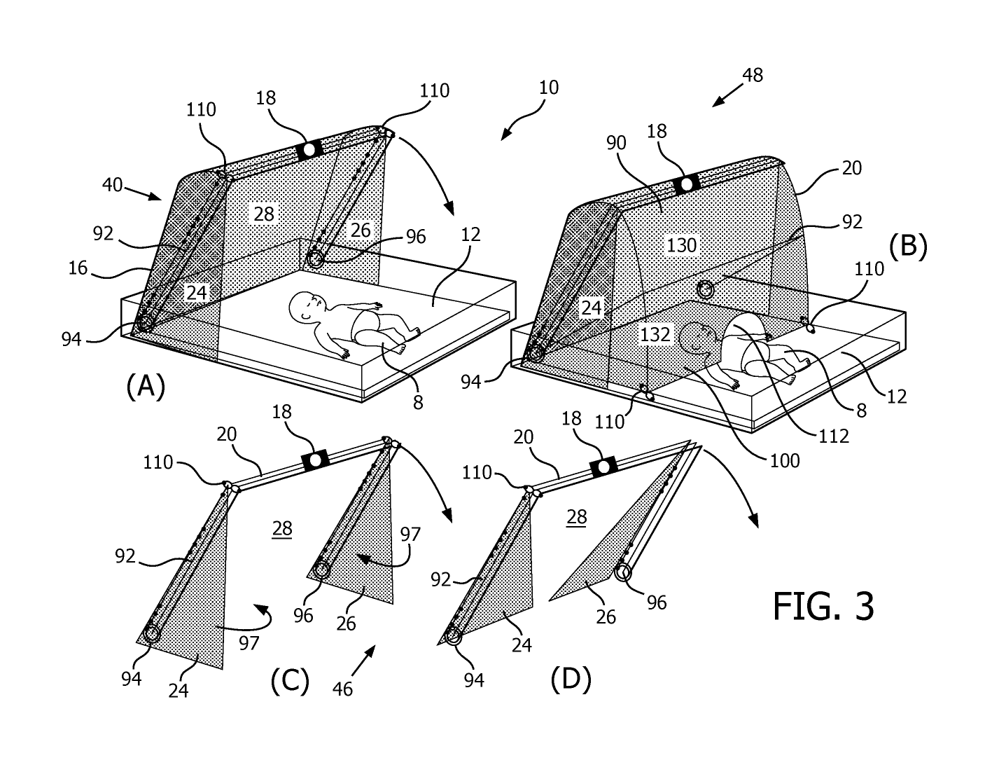

FIG. 3 illustrates a collapsible embodiment of infant sleeping surface attachment system 10. System 10 may be collapsed when not in use. Collapsing system 10 may make it less bulky, easier to transport, easier to use in a home environment, easier to store away when not in use, and/or may have other advantages. FIG. 3A and FIG. 3B illustrate infant sleeping surface 12 with fixed portion 16 and cover portion 20 in a retracted position (FIG. 3A) and a closed position (FIG. 3B). FIG. 3C and FIG. 3D illustrate collapsing system 10.

In some embodiments, collapsible system 10 does not include base panel 22. As shown in FIG. 3, in some embodiments, at third side 50, first side panel 24 and second side panel 26 may not have corresponding arcuately shaped edges 68 (as shown in FIGS. 1A and 1B). For example, first side panel 24 and second side panel 26 may be generally triangularly shaped. End panel 28 is coupled with first side panel 24 and second side panel 26 at first end 40 of infant sleeping surface 12. In these embodiments, first side panel 24 and second side panel 26 may be pivotally coupled with end panel 28. First side panel 24 and/or second side panel 26 may be pivotally coupled with end panel 28 via hinges, and or other pivoting coupling mechanisms. A user may collapse system 10 by retracting cover portion 20, then folding 97 side panels 24 and 26 inward toward cavity 30. In some embodiments cover portion 20 may be decoupled from side panes 24 and 26 at bearings 94 and 96 prior to collapsing system 10. In some embodiments, cover portion 20 may remain coupled with fixed portion 16 during collapse. In some embodiments, as described above, cover portion 20 is retracted into cavity 30. In some embodiments, cover portion 20 and fixed portion 16 may be configured such that cover portion 20 is coupled to (e.g., via hinges 94 and 96) outside surfaces of first side panel 24 and second side panel 26 (e.g., facing toward first side 46 and second side 48 respectively, away from cavity 30). When cover portion 20 is coupled to the outside surfaces first side panel 24 and second side panel 26, cover portion 20 may retract around fixed portion 16 (not into cavity 30).

In some embodiments, collapsible system 10 includes base panel 22. In these embodiments, base panel 22 may be removably coupled with first side panel 24 and/or second side panel 26. In some embodiments, base panel 22 may be removably coupled with first side panel 24 and/or second side panel 26 by one or more removable coupling mechanisms located along the corresponding edges of base panel 22 and first side panel 24 and/or based panel 22 and/or second side panel 26. The one or more removable coupling mechanisms may include magnets, hooks, clamps, straps, fasteners, and/or other removable coupling mechanisms. Base panel 22 may be pivotally coupled with end panel 28. A user may collapse system 10 by retracting cover portion 20, decoupling base panel 22 from first side panel 24 and second side panel 26, folding side panels 24 and 26 inward toward cavity 30, and then folding base panel 22 toward end panel 28.

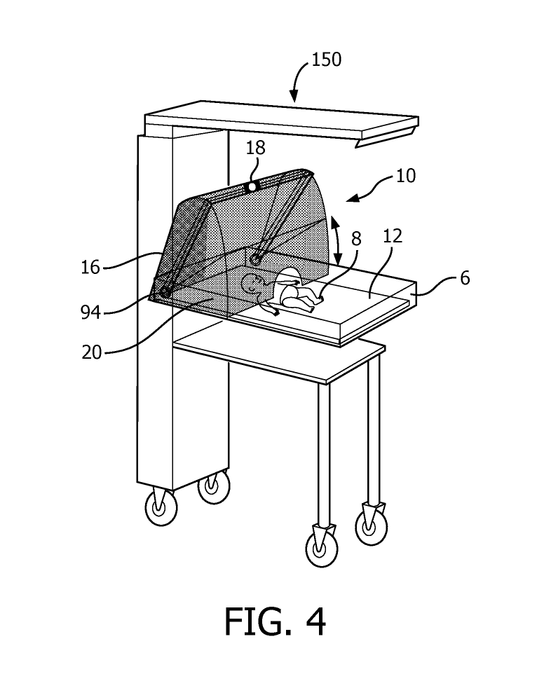

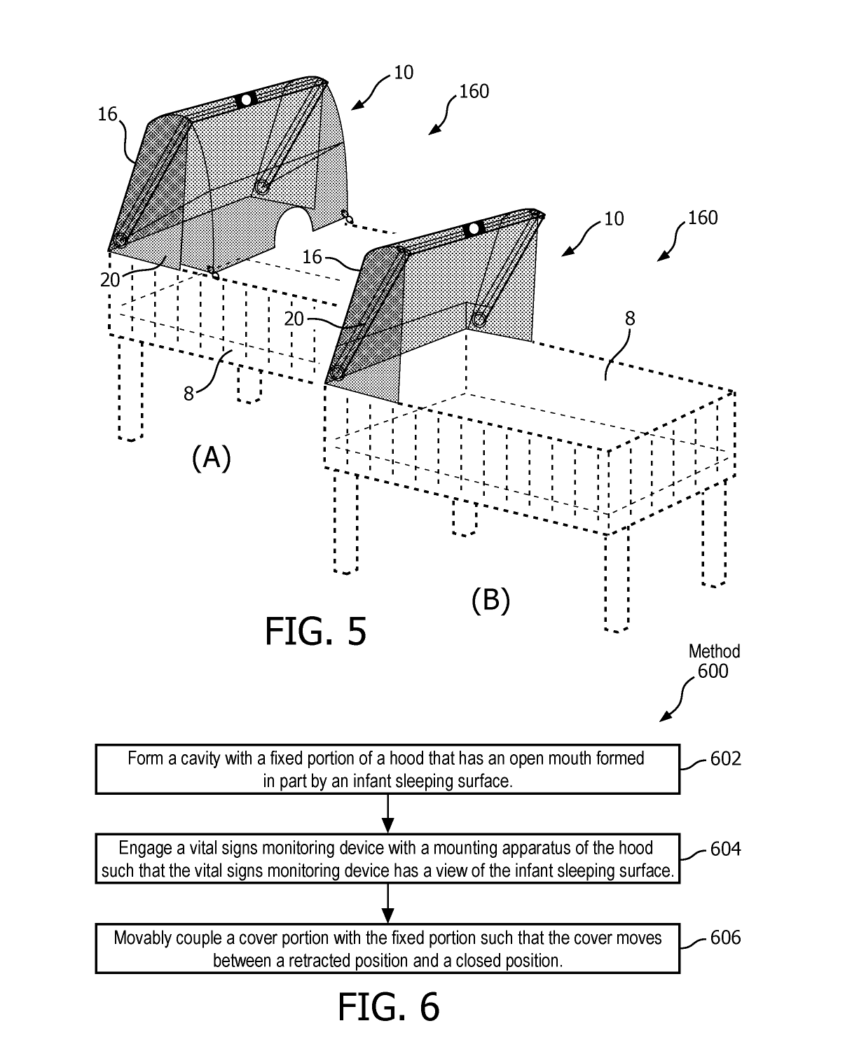

FIG. 4 and FIG. 5 illustrate system 10 being used in different situations. FIG. 4 illustrates system 10 on a typical warmer 150 in a NICU. Cover portion 20 may be retracted to allow a maximum amount of warming for infant 8 and then closed when oxygen therapy is necessary. Mounting apparatus 18 is configured to engage a vital signs monitoring device so the vital signs monitoring device has a view of infant sleeping surface 12 during the time infant 8 is on warmer 150. If necessary, system 10 may be easily removed from infant sleeping surface 12 and/or warmer 150.

FIG. 5 illustrates system 10 in use at home (FIG. 5B) and/or in a ward at a hospital (FIG. 5A). System 10 may be held engaged in a fixed position relative to a mattress in a cradle 160, for example, and/or other infant beds. FIG. 5A illustrates system 10 in the closed position. FIG. 5B illustrates system 10 in the retracted position. Collapsible embodiments of system 10 may be useful for home use and/or in hospital wards. However, the illustrated uses of system 10 in FIG. 4 and FIG. 5 are not intended to be limiting. System 10 may be used in any situation where the advantages of system 10 are useful (e.g., collapsible embodiments and/or non-collapsible embodiments).

FIG. 6 illustrates a method 600 for facilitating vital signs monitoring of an infant laying on an infant sleeping surface with an infant sleeping surface attachment system. In some embodiments (e.g., such as when the infant sleeping surface attachment system is used in a hospital NICU setting), method 600 includes delivering oxygen to an infant laying in the infant sleeping surface. The system includes a hood, a cover portion, and/or other components. The hood includes a fixed portion, a mounting apparatus, and/or other components. The operations of method 600 presented below are intended to be illustrative. In some embodiments, method 600 may be accomplished with one or more additional operations not described, and/or without one or more of the operations discussed. Additionally, the order in which the operations of method 600 are illustrated in FIG. 6 and described below is not intended to be limiting.

At an operation 602, a cavity is formed. The cavity is formed by the fixed portion. The fixed portion is held engaged in a fixed position relative to the infant sleeping surface. The cavity has an open mouth formed in part by the infant sleeping surface. In some embodiments, operation 602 is performed by a fixed portion the same as or similar to fixed portion 16 (shown in FIG. 1 and described herein).

At an operation 604, a vital signs monitoring device is engaged. The vital signs monitoring device is engaged with the mounting apparatus. The mounting apparatus is coupled to the fixed portion inside the cavity. The mounting apparatus is configured to carry the vital signs monitoring device so the vital signs monitoring device has a view of the infant sleeping surface. In some embodiments, operation 604 is performed by a mounting apparatus the same as or similar to mounting apparatus 18 (shown in FIG. 1 and described herein).

At an operation 606, the cover portion movably couples with the fixed portion. The flexible cover portion movably couples with the fixed portion such that the cover portion moves and/or slides with respect to the fixed portion between a retracted position and a closed position. In the retracted position, the flexible cover portion is folded. In the retracted position, the cover portion permits physical access to the cavity. In the closed position, the cover portion covers the open mouth of the cavity. In the closed position the flexible cover portion is unfolded like an arc (e.g., like a pram hood). In some embodiments, operation 606 is performed by a cover portion the same as or similar to cover portion 20 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. An infant sleeping surface attachment system configured to facilitate vital signs monitoring of an infant laying on an infant sleeping surface, the system comprising:
a hood, the hood comprising:
a fixed portion configured to be held engaged in a fixed position relative to the infant sleeping surface, the fixed portion forming a cavity having an open mouth formed in part by the infant sleeping surface;
wherein the fixed portion includes a mounting apparatus configured to engage a vital signs monitoring device, the mounting apparatus coupled to the fixed portion inside the cavity, the mounting apparatus configured to carry the vital signs monitoring device so the vital signs monitoring device has a view of the infant sleeping surface; and
wherein the fixed portion comprises a fixing assembly configured to retain a relative position of the fixed portion and the mounting apparatus to the infant sleeping surface; and
a cover portion, formed at least in part from a flexible material, that is movably coupled with the fixed portion such that the cover portion moves with respect to the fixed portion between a retracted position and a closed position, wherein, in the closed position, the cover portion covers the open mouth of the fixed portion, and wherein, in the retracted position, the cover portion permits physical access to the cavity.

2. The system of claim 1, wherein the fixing assembly of the fixed portion includes a base panel configured to be positioned under the infant sleeping surface.

3. The system of claim 1, wherein the infant sleeping surface attachment system is collapsible.

4. The system of claim 1, wherein the vital signs monitoring device is a vital sign camera, and wherein the mounting apparatus is configured such that the vital sign camera has a view of a face of an infant laying on the infant sleeping surface and an angled view of the chest of such an infant.

5. The system of claim 1, wherein the hood and the cover portion are formed from substantially transparent materials, and wherein, responsive to the cover portion being in the closed position, the infant sleeping surface attachment system forms a flexible hood that is utilized with a supply of oxygen as an oxyhood for delivering oxygen to the infant.

6. A method for facilitating vital signs monitoring of an infant laying on an infant sleeping surface with an infant sleeping surface attachment system, the system comprising a hood and a cover portion that is formed at least in part from a flexible material, the hood comprising a fixed portion and a mounting apparatus, the method comprising:
forming a cavity with the fixed portion that is held engaged in a fixed position relative to the infant sleeping surface, the cavity having an open mouth formed in part by the infant sleeping surface;
engaging a vital signs monitoring device with the mounting apparatus, the mounting apparatus coupled to the fixed portion inside the cavity, the mounting apparatus configured to carry the vital signs monitoring device so the vital signs monitoring device has a view of the infant sleeping surface; and
retaining, with a fixing assembly of the fixed portion, a relative position of the fixed portion and the mounting apparatus to the infant sleeping surface;
movably coupling the cover portion with the fixed portion such that the cover portion moves with respect to the fixed portion between a retracted position and a closed position, wherein, in the closed position, the cover portion covers the open mouth of the cavity, and wherein, in the retracted position, the cover portion permits physical access to the cavity.

7. The method of claim 6, wherein the fixing assembly of the fixed portion includes a base panel and wherein the method further comprises positioning the base panel under the infant sleeping surface.

8. The method of claim 6, further comprising collapsing the infant sleeping surface attachment system.

9. The method of claim 6, wherein the vital signs monitoring device is a vital signs camera, and wherein the method further comprises engaging the vital signs camera with the mounting apparatus such that the vital signs camera has a view of a face of an infant laying on the infant sleeping surface and an angled view of the chest of such an infant.

10. The method of claim 6, further comprising forming the hood and the cover portion from substantially transparent materials, and wherein, responsive to the cover portion covering the open mouth of the cavity, the infant sleeping surface attachment system forms a flexible hood that is utilized with a supply of oxygen as an oxyhood for delivering oxygen to the infant.

11. An infant sleeping surface attachment system configured to facilitate vital signs monitoring of an infant laying on an infant sleeping surface, the system comprising:
means for forming a cavity that is held engaged in a fixed position relative to the infant sleeping surface, the cavity having an open mouth formed in part by the infant sleeping surface;
means for engaging a vital signs monitoring device, the means for engaging coupled to the means for forming a cavity inside the cavity, the means for engaging configured to carry the vital signs monitoring device so the vital signs monitoring device has a view of the infant sleeping surface;
means for retaining a relative position of the means for forming a cavity and the means for engaging to the infant sleeping surface; and
means for covering the open mouth of the cavity and permitting physical access to the cavity formed at least in part from a flexible material, the means for covering and permitting configured to move with respect to the means for forming a cavity between a retracted position and a closed position, and wherein, in the retracted position, the means for covering and permitting permits physical access to the cavity, and in the closed position, covers the open mouth of the cavity.

12. The system of claim 11, wherein the means for retaining a relative position includes a base panel configured to be positioned under the infant sleeping surface.

13. The system of claim 11, wherein the infant sleeping surface attachment system is collapsible.

14. The system of claim 11, wherein the vital signs monitoring device is a vital signs camera, and wherein the means for engaging is configured such that the vital signs camera has a view of a face of an infant laying on the infant sleeping surface and an angled view of the chest of such an infant.

15. The system of claim 11, wherein the means for forming a cavity and the means for covering and permitting are formed from substantially transparent materials and wherein, responsive to the means for covering and permitting being in the closed position, the infant sleeping surface attachment system forms a flexible hood that is utilized with a supply of oxygen as an oxyhood for delivering oxygen to the infant.

16. The system of claim 1, wherein the vital signs monitoring device is a vital sign camera.

17. The system of claim 16, wherein the mounting apparatus is configured to engage the vital sign camera so that the vital sign camera is positioned fully within the cavity such that the vital sign camera has a substantially uninterrupted view of the infant sleeping surface when the cover is in the retracted position and the closed position.

18. The system of claim 2, wherein the fixing assembly of the fixed portion includes a first side panel and a second side panel coupled with the base panel.

19. The system of claim 18, wherein the fixing assembly of the fixed portion includes an end panel coupled with the base panel, the first side panel, and the second side panel.

20. The method of claim 6, wherein the vital signs monitoring device is a vital sign camera.

21. The method of claim 20, wherein the method further comprises engaging the vital sign camera so that the camera is positioned fully within the cavity such that the vital sign camera has a substantially uninterrupted view of the infant sleeping surface when the cover is in the retracted position and the closed position.

22. The method of claim 7, further comprising coupling a first side panel and a second side panel of the fixing assembly with the base panel.

23. The method of claim 22, further comprising coupling an end panel of the fixing assembly with the base panel, the first side panel, and the second side panel.

24. The system of claim 11, wherein the vital signs monitoring device is a vital sign camera.

25. The system of claim 24, wherein the means for engaging is coupled to the means for forming a cavity inside the cavity such that the vital sign camera is positioned fully within the cavity and has a substantially uninterrupted view of the infant sleeping surface when the means for covering is in the retracted position and the closed position.

26. The system of claim 12, wherein the means for retaining a relative position includes a first side panel and a second side panel coupled with the base panel.

27. The system of claim 26, wherein the means for retaining a relative position includes an end panel coupled with the base panel, the first side panel, and the second side panel.
